(19) United States
(12) Patent Application Publication
(10) Pub. No.: US 2014/0143905 A1
(43) Pub. Date: May 22, 2014

(54) MAIZE PLANTS HAVING A PARTIALLY OR FULLY MULTIPLIED GENOME AND USES THEREOF

(71) Applicants: Amit Avidov, Kiryat-Tivon (IL); Alon Lerner, Moshav Sharona (IL); Limor Baruch, Moshav Tel Adashim (IL)

(72) Inventors: Amit Avidov, Kiryat-Tivon (IL); Alon Lerner, Moshav Sharona (IL); Limor Baruch, Moshav Tel Adashim (IL)

(21) Appl. No.: 14/233,789

(22) PCT Filed: Jul. 17, 2012
(86) PCT No.: PCT/IL2012/050252

§ 371 Date: Jan. 20, 2014

Related U.S. Application Data
(60) Provisional application No. 61/509,633, filed on Jul. 20, 2011.

(51) Int. Cl. A01H 5/10; A01H 1/08; A01H 1/02

(52) U.S. Cl. CPC A01H 5/10; A01H 1/02; A01H 1/08
USPC 800/275; 800/320.1; 554/9; 435/412; 800/276; 44/307; 426/622

(57) ABSTRACT

A maize plant or plant part having a partially or fully multiplied genome is provided. Also provided are methods of generating and using the plant or parts thereof, and products comprising same.

### FIELD AND BACKGROUND OF THE INVENTION

The present invention, in some embodiments thereof, relates to maize plants having a partially or fully multiplied genome and uses thereof.

Maize (Zea mays L. ssp) also known as corn or mielie/mealie, is a grain domesticated by indigenous peoples in Mesoamerica in prehistoric times. The leafy stalk produces ears which contain seeds called kernels. Maize kernels are technically a fruit but are used in cooking as a vegetable or starch. Maize has 10 chromosomes (n=10). The combined length of the chromosomes is 1500 cM. Some of the maize chromosomes have what are known as “chromosomal knobs”: highly repetitive heterochromatic domains that stain darkly. Individual knobs are polymorphic among strains of both maize and teosinte.

Maize is the most widely produced feed grain in the United States, accounting for more than 90 percent of total production. Around 80 million acres of land are planted with maize. The majority of the crop is used as livestock feed; the remainder is processed into a multitude of food and industrial products including starch, sweeteners such as high fructose corn syrup, corn oil, and ethanol for use as a fuel.

Maize and cornmeal (ground dried maize) constitute a staple food in many regions of the world. Introduced into Africa by the Portuguese in the 16th century, maize has become Africa's most important staple food crop. Maize meal is also used as a replacement for wheat flour, to make cornbread and other baked products. Maize is a major source of starch. Cornstarch (maize flour) is a major ingredient in home cooking and in many industrialized food products. Maize is also a major source of cooking oil (corn oil) and of maize gluten. Maize starch can be hydrolyzed and enzymatically treated to produce syrups, particularly high fructose corn syrup, a sweetener; and also fermented and distilled to produce grain alcohol. Maize is sometimes used as the starch source for beer. Within the United States, the usage of maize for human consumption constitutes about 1/40th of the amount grown in the country. In the United States and Canada maize is mostly grown as feed for livestock, as forage, silage (made by fermentation of chopped green cornstalks), or grain. Maize meal is also a significant ingredient of some commercial animal food products.

Attempts to increase maize yields using classical breeding, genetic engineering and other agricultural practices are well known in the art.

For example, production of biomass in the form of grain and stover has conventionally been increased by the use of fertilizers, pesticides and selective breeding. Although the application of fertilizers and the elimination of insect pests have resulted in increased biomass, the effectiveness of all these methods is limited by the genetic makeup of the plant. Conversely, selective breeding often times ends with reduction in performance, yield, and plant size as a result of inbreeding depression.

Genetically modified (GM) maize is one of the 11 GM crops grown commercially in 2009. Grown since 1997 in the United States and Canada, 85% of the US maize crop was genetically modified in 2009. It is also grown commercially in Brazil, Argentina, South Africa, Canada, the Philippines, Spain and, on a smaller scale, in the Czech Republic, Portugal, Egypt and Honduras. However, the use of genetically engineered maize in the United States for more than a decade has had little impact on crop yields despite claims that they could ease looming food shortages.

### RELATED ART

U.S. Patent Application 20090162477
U.S. Patent Application 20030005479
U.S. Pat. No. 4,705,910

### ADDITIONAL BACKGROUND ART

Dudley and Alexander 1969 Crop Science 9:613-615;
Randolph Proc. N.A.S. 18:222-229 1923;
Randolph Agricultural Research 1935 50(7):591-605;
Randolph Agricultural Research 1944 69:47-76;
Rice and Dudley 1974 Crop Science 14:390-393

### SUMMARY OF THE INVENTION

According to an aspect of some embodiments of the present invention there is provided a maize (Zea mays L. ssp) plant having a partially or fully multiplied genome as exemplified herein.

According to an aspect of some embodiments of the present invention there is provided a maize plant having at least 43 chromosomes and being at least as fertile as a diploid maize (Zea mays L. ssp) plant isogenic thereto when being of the same developmental stage and when grown under the same conditions.

According to an aspect of some embodiments of the present invention there is provided a maize plant having a partially or fully multiplied genome and characterized by a seed weight at least 10% higher than that of a diploid maize (Zea mays L. ssp) plant isogenic thereto, when being of the same developmental stage and when grown under the same conditions.

According to an aspect of some embodiments of the present invention there is provided a maize plant having a partially or fully multiplied genome and characterized by a total dry weight at least 30% higher than that of a diploid maize (Zea mays L. ssp) plant isogenic thereto when being of the same developmental stage and when grown under the same conditions.

According to an aspect of some embodiments of the present invention there is provided a hybrid plant having as a parental ancestor the plant of the invention.

According to an aspect of some embodiments of the present invention there is provided a planted field comprising the plant of the invention.

According to an aspect of some embodiments of the present invention there is provided a sown field comprising seeds of the plant of the invention.

According to some embodiments of the invention, the plant of the invention has a seed weight at least 10% higher than that of the diploid maize (Zea mays L. ssp) plant isogenic thereto when being of the same developmental stage and when grown under the same conditions.

According to some embodiments of the invention, the plant of the invention has a total dry weight at least 30% higher than that of the diploid maize (Zea mays L. ssp) plant isogenic thereto when being of the same developmental stage and when grown under the same conditions.

According to some embodiments of the invention, the plant of the invention exhibits higher CO₂ uptake/per unit leaf area than that of the diploid maize (Zea mays L. ssp) plant isogenic thereto when being of the same developmental stage and when grown under the same conditions.

According to some embodiments of the invention, the plant of the invention is at least as fertile as the diploid maize (Zea mays L. ssp) plant isogenic thereto when being of the same developmental stage and when grown under the same conditions.

According to some embodiments of the invention, the plant of the invention is non-transgenic.

According to some embodiments of the invention, the fertility is determined by at least one of:

number of seeds per plant;

seed set assay;

gamete fertility assay; and

acetocarmine pollen staining.

According to some embodiments of the invention, the plant of the invention is a triploid;

According to some embodiments of the invention, the plant of the invention is a tetraploid.

According to some embodiments of the invention, the plant of the invention is capable of cross-breeding with a diploid or tetraploid maize.

According to some embodiments of the invention, the plant of the invention is an inbred.

According to an aspect of some embodiments of the present invention there is provided a plant part of the plant of the invention.

According to some embodiments of the invention, the plant part is a seed, or a grain.

According to an aspect of some embodiments of the present invention there is provided a processed product of the plant of the invention or the plant part.

According to some embodiments of the invention, the processed product is selected from the group consisting of food, feed, herbal supplement, beverages, adhesive, construction material, biodiesel and biofuel.

According to some embodiments of the invention, the food or feed is selected from the group consisting of silage, hominy, corn flakes, polenta and popcorn.

According to an aspect of some embodiments of the present invention there is provided a meal produced from the plant of the invention or the plant part.

According to an aspect of some embodiments of the present invention there is provided a method of producing oil, the method comprising:

(a) harvesting grains of the plant; and

(b) extracting oil from the grains.

According to some embodiments of the invention, the method further comprises processing the oil into biodiesel.

According to an aspect of some embodiments of the present invention there is provided an isolated regenerable cell of the plant of the invention or the plant part.

According to some embodiments of the invention, the cell exhibits genomic stability for at least 5 passages in culture.

According to some embodiments of the invention, the cell is from a mertistem, a pollen, a leaf, a root, a root tip, an anther, a pistil, a flower, a seed, a grain, a straw or a stem.

According to an aspect of some embodiments of the present invention there is provided a tissue culture comprising the regenerable cells.

According to an aspect of some embodiments of the present invention there is provided a method of producing maize seeds, comprising self-breeding or cross-breeding the plant of the invention.

According to an aspect of some embodiments of the present invention there is provided a method of developing a hybrid plant using plant breeding techniques, the method comprising using the plant of the invention as a source of breeding material for self-breeding and/or cross-breeding.

According to an aspect of some embodiments of the present invention there is provided a method of producing maize meal, the method comprising:

(a) harvesting grains of the plant or plant part; and

(b) processing the grains to produce the maize meal.

According to an aspect of some embodiments of the present invention there is provided a method of generating a maize seed having a partially or fully multiplied genome, the method comprising contacting the maize seed with a G2/M cell cycle inhibitor under a transiently applied magnetic field thereby generating the maize seed having a partially or fully multiplied genome.

According to some embodiments of the invention, the G2/M cell cycle inhibitor comprises a microtubule polymerization inhibitor.

According to some embodiments of the invention, the microtubule polymerization inhibitor is selected from the group consisting of colchicine, nocodazole, oryzaline, trifluraline and vinblastine sulphate.

According to some embodiments of the invention, the method further comprises sonicating the seed prior to contacting.

According to an aspect of some embodiments of the present invention there is provided a sample of representative seeds of a maize plant having at least 43 chromosomes and being at least as fertile as a diploid maize (Zea mays L. ssp) plant isogenic thereto when being of the same developmental stage and when grown under the same conditions, wherein the sample has been deposited under the Budapest Treaty at the NCIMB under NCIMB 41973.

According to an aspect of some embodiments of the present invention there is provided a sample of representative seeds of a maize plant having at least 43 chromosomes and being at least as fertile as a diploid maize (Zea mays L. ssp) plant isogenic thereto when being of the same developmental stage and when grown under the same conditions, wherein the sample of the maize plant having at least 43 chromosomes has been deposited under the Budapest Treaty at the NCIMB under NCIMB 41973.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing thousand seed weight (gr) of the indicated diploid and triploid hybrids as well as that of the female diploid parent.

FIG. 2 is a graph showing cumulative photosynthesis in the diploid (EXPM100) and triploid (EXPM104) maize of some embodiments of the present invention;

FIGS. 3A-B are images of maize seeds generated according to the teachings of the present invention. FIG. 3A shows seeds of a male diploid parent and seeds of the tetraploid male generated by genome multiplication of the diploid male. FIG. 3B shows the seeds of diploid and triploid hybrids.

FIGS. 4A-B are graphs showing thousand seed weight (gr) of the indicated diploid and triploid hybrids as well as the female diploid parent.

FIGS. 5A-B are graphs of maize seeds generated according to the teachings of the present invention.

FIG. 6 is a graph showing cumulative photosynthesis in the diploid and tetraploid maize of some embodiments of the present invention;

FIGS. 7A-D are images of hybrid diploid and triploid maize seeds.

### DESCRIPTION OF SPECIFIC EMBODIMENTS OF THE INVENTION

The present invention, in some embodiments thereof, relates to maize (Zea mays L. ssp) plants having a partially or fully multiplied genome and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Maize (Zea mays L.) and cornmeal (ground dried maize) constitute a staple food in many regions of the world. As the world population continues to grow geometrically, great pressure is being placed on increasing plant yield.

Selective breeding has been employed for centuries to improve, or attempt to improve, phenotypic traits of agronomic and economic interest in plants such as yield, percentage of grain oil, etc. Generally speaking, selective breeding involves the selection of individuals to serve as parents of the next generation on the basis of one or more phenotypic traits of interest. However, such phenotypic selection is frequently complicated by non-genetic factors that can impact the phenotype(s) of interest. Likewise, attempts to increase crop yield by genetic engineering have resulted in only marginal success.

The present inventors have now designed a novel procedure for induced genome multiplication in maize (Zea mays L. ssp.) that results in plants which are genomically stable and fertile. The polyploid plants (e.g., tetraploid and triploid) are devoid of undesired genomic mutations and are characterized by stronger vigor and higher total plant yield than that of the isogenic progenitor plant having a diploid genome (see Table 5, below). These new traits may contribute to better climate adaptability and higher tolerance to biotic and abiotic stress. Furthermore, hybrid maize seeds (or grains, as interchangeably used herein) generated by pollen sterilization using the induced polyploid plants of the present invention may increase global corn yield by dozens of percents due to heterosis expression. In addition, the polyploid plant of some embodiments of the invention exhibits comparable or better fertility to that of the isogenic diploid progenitor plant already from early generations (e.g., first, second, third or fourth) following genome multiplication, negating the need for further breeding in order to improve fertility.

Thus, according to an aspect of the invention there is provided a maize plant having at least 43 chromosomes and being at least as fertile as a diploid maize (Zea mays L. ssp.) plant isogenic thereto when being of the same developmental stage and when grown under the same conditions.

According to an additional or alternative aspect there is provided a plant having a partially or fully multiplied genome and characterized by a seed weight at least 10% higher than that of a diploid maize (Zea mays L. ssp.) plant isogenic thereto, when being of the same developmental stage and when grown under the same conditions.

According to an additional or alternative aspect there is provided a maize plant having a partially or fully multiplied genome and characterized by a total dry weight at least 30% higher than that of a diploid maize (Zea mays L. ssp.) plant isogenic thereto when being of the same developmental stage and when grown under the same conditions.

As used herein, the term “maize (Zea mays L. ssp.) plant” refers to the corn that is conventionally grown for human or animal food or beverages or as a source of raw materials, food supplements or chemicals. The maize plant is a diploid (2N=20) in nature.

A number of commercial varieties are available including, but not limited to:

Zea mays var. amylacea (typically used for producing corn flower)

Zea mays var. everta (typically used for producing pop-corn)

Zea mays var. indentata (Dent corn)

Zea mays var. indurata (Flint corn)

Zea mays var. saccharata and Zea mays var. rugosa (Sweet corn)

Zea mays var. ceratin (Waxy corn)

Zea mays (Amylomaize)

Zea mays var. tunicata Larrañaga ex A. St. Hil (Pod corn)

Zea mays var. japonica (Striped maize)

“A plant” refers to a whole plant or portions thereof (e.g., seeds, and stover e.g., stems, straw, leaves, tissues etc.), processed or non-processed (e.g., seeds, meal, stems, dry tissue, cake, oil etc.), regenerable tissue culture or cells isolated therefrom.

According to some embodiments, the term plant as used herein also refers to hybrids having one of the induced polyploid plants as at least one of its ancestors, as will be further defined and explained hereinbelow.

As used herein “partially or fully multiplied genome” refers to an addition of at least one chromosome, a partial chromosome set (<10), a chromosome set (N=10) or a full multiplication of the genome that results in a tetraploid plant (4N) or more.

According to a specific embodiment, the polyploid plant is 3N.

According to a specific embodiment, the polyploid plant is 4N.

According to a specific embodiment, the polyploid plant is 5N.

According to a specific embodiment, the polyploid plant is 6N.

According to a specific embodiment, the polyploid plant is 7N.

According to a specific embodiment, the polyploid plant is 8N.

According to a specific embodiment, the polyploid plant is 9N.

According to a specific embodiment, the polyploid plant is 10N.

According to a specific embodiment, the polyploid plant is 11N.

According to a specific embodiment, the polyploid plant is 12N.

According to a specific embodiment, the induced polyploid plant is not a genomically multiplied haploid plant.

As mentioned multiplication can result in the addition of an incomplete chromosome set (<1N) or alternatively in the addition of an incompletely multiplied chromosome sets.

Thus, according to a specific embodiment, the polyploid plant of the present invention comprises at least 43 chromosomes.

As mentioned, the polyploid plant is at least as fertile as the diploid maize (Zea mays L. ssp.; N=20) plant isogenic thereto, when being of the same (identical) developmental stage and when grown under the same conditions.

As used herein the term “fertile” refers to the ability to reproduce sexually. Fertility can be assayed using methods which are well known in the art. Alternatively, fertility is defined as the ability to set seeds. The following parameters may be assayed in order to determine fertility: the number of seeds (grains); seed set assay; gamete fertility may be determined by pollen germination such as on a sucrose substrate; and alternatively or additionally acetocarmine staining, whereby a fertile pollen is stained.

As mentioned, the polyploid plant of some embodiments of the invention exhibits comparable fertility (e.g., +/− about 10% or 20%) to that of the isogenic diploid progenitor plant already from early generations (e.g., first, second, third or fourth) following genome multiplication, negating the need for further breeding.

As used herein the term “stable” or “genomic stability” refers to the number of chromosomes or chromosome copies, which remains constant through several generations, while the plant exhibits no substantial decline in at least one of the following parameters: yield, fertility, biomass and vigor. According to a specific embodiment, stability is defined as producing a true to type offspring, keeping the variety strong and consistent.

According to an embodiment of the invention, the genomically multiplied plant is isogenic to the source plant, namely the diplod maize. The genomically multiplied plant has substantially the same genomic composition as the diploid plant in quality but not in quantity.

According to a specific embodiment, the plant (or a cell culture derived therefrom) exhibits genomic stability for at least 2, 3, 5, 10 or more passages in culture or generations. According to some embodiments of the present invention, a mature genomically multiplied plant has at least about the same (+/−10%) number of seeds as it's isogenic diploid progenitor grown under the same conditions and being of the same developmental stage; alternatively or additionally the genomically multiplied plant has at least 90% fertile pollen that are stained by acetocarmine; and alternatively or additionally at least 90% of seeds germinate on sucrose.

According to some embodiments of the present invention, the polyploid plant has a seed weight at least 7%, 10%, 20%, 30%, 40%, 50%, 60%, 70%, 80%, 90%, 100%, 120%, 150% or 200% higher than that of the diploid maize plant isogenic thereto when being of the same developmental stage and when grown under the same conditions.

Seed weight can be measured for a quota of seeds (e.g., 1000) or per single seed.

According to some embodiments of the present invention, the polyploid plant has a total dry weight at least 20%, 25%, 30%, 40%, 50%, 60%, 70%, 80%, 90%, 100%, 120%, 150% or 200% higher than that of the diploid maize plant isogenic thereto when being of the same developmental stage and when grown under the same conditions.

Dry weight is used as a measure of plant growth. The plant is removed from the soil and washed of any loose soil. The plant is dried in an oven set to low heat (e.g., 100° C.) overnight. The plant is cooled in a dry environment and then weighed.

According to some embodiments of the present invention, the polyploid plant exhibits higher CO₂ uptake (per unit leaf area, 10%, 20%, 50%, 70% or higher as determined by CO₂ uptake assay described in the Examples section that follows) than that of the diploid maize plant isogenic thereto when being of the same developmental stage and when grown under the same conditions. Methods of measuring CO₂ uptake as a measure of plant growth rate are known in the art and described in the Examples section which follows.

Comparison assays done for characterizing traits (e.g., fertility, yield, biomass and vigor) of the genomically multiplied plants of the present invention are typically affected in comparison to it's isogenic progenitor (hereinafter, “the diploid progenitor plant”) when both are being of the same developmental stage and both are grown under the same growth conditions.

According to a specific embodiment, the genomically multiplied plant is characterized by grain protein content at least as similar to that of the diploid maize isogenic progenitor plant of the same developmental stage and grown under the same growth conditions. According to a specific embodiment the grain protein content is higher or lower by about 0-20% of that of the isogenic progenitor plant of the same developmental stage and grown under the same growth conditions.

According to a specific embodiment, the genomically multiplied plant is characterized by a grain yield per growth area at least as similar to that of the diploid maize isogenic progenitor plant of the same developmental stage and grown under the same growth conditions. According to a specific embodiment the grain yield per growth area is higher by 10%, 15%, 20%, 25%, 30%, 35%, 40%, 45%, 50% or even more 80%, 90%, 100%, 200, %, 250%, 300%, 400% or 500%. According to a specific embodiment the grain yield per growth area is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by a grain yield per plant at least as similar to that of the diploid maize isogenic progenitor plant of the same developmental stage and grown under the same growth conditions. According to a specific embodiment the grain yield per plant is higher by 5%, 10%, 15%, 20%, 25%, 30%, 35%, 40%, 45%, 50% or even more 80%, 90%, 100%, 200, %, 250%, 300%, 400% or 500%. According to a specific embodiment the grain yield per plant is higher by 0.1-5, 0.3-5, 0.4-2.5, 1-5, 2-3 or 2-2.5 fold than that of the isogenic progenitor plant of the same developmental stage and grown under the same growth conditions.

Plants of the invention are characterized by at least one, two, three, four or all of higher biomass, yield, grain yield, grain yield per growth area, grain protein content, grain weight, stover yield, seed set, chromosome number, genomic composition, percent oil, vigor, insect resistance, pesticide resistance, drought tolerance, and abiotic stress tolerance than the diploid plant isogenic thereto.

According to a specific embodiment there is provided a maize plant having a partially or fully multiplied genome as exemplified herein.

According to a specific embodiment, the plant is a polyploid as exemplified herein.

Thus according to an embodiment, the tetraploid plant is Z1(13)-2011 or 21(12)-2011 having the Z1-2011 diploid as the isogenic progenitor.

Using the present teachings, the present inventors were able to generate a number of plant varieties which are induced polyploids. A sample of representative seeds of a maize plant having at least 43 chromosomes and being at least as fertile as a diploid maize (Zea mays L. ssp) plant isogenic thereto when being of the same developmental stage and when grown under the same conditions, wherein a sample of the maize plant having at least 43 chromosomes has been deposited under the Budapest Treaty at the NCIMB under NCIMB 41973 on May 18, 2012.

Hybrid triploids of Z1(13)-2011 or Z1(12)-2011 are also provided where the female parent is N2-2011. Thus, hybrid triploid HF1 code EXPM 104 has as the parental male tetraploid Z1(13)-2011; Hybrid triploid HF1 code EXPM 110 has as the parental male tetraploid Z1(12)-2011.

According to another embodiment, the tetraploid plant is Z5(6)-2011 or Z5(8)-2011 having the Z5-2011 diploid as the isogenic progenitor. Hybrid triploids of Z5(6)-2011 or Z5(8)-2011 are also provided where the female parent is N2-2011.

Thus, hybrid triploid HF1 code EXPM 208 has as the parental male tetraploid Z5(6)-2011; Hybrid triploid HF1 code EXPM 211 has as the parental male tetraploid Z5(8)-2011.

According to another embodiment, the tetraploid plant is Z5(39)-2011, Z5(22)-2011, Z5(8)-2011, or Z5(31)-2011 having the Z5-2011 diploid as the isogenic progenitor.

Hybrid triploids of Z5(39)-2011, Z5(22)-2011, Z5(8)-2011, or Z5(31)-2011 are also provided where the female parent is N5-2011.

Thus, hybrid triploid HF1 code EXPM 303 has as the parental male tetraploid Z5(39)-2011; Hybrid triploid HF1 code EXPM 307 has as the parental male tetraploid Z5(22)-2011; Hybrid triploid HF1 code EXPM 309 has as the parental male tetraploid Z5(8)-2011; Hybrid triploid HF1 code EXPM 310 has as the parental male tetraploid Z5(31)-2011.

According to a specific embodiment, the plant is non-transgenic.

According to another embodiment, the plant is transgenic for instance by expressing a heterologous gene conferring pest resistance or morphological traits for cultivation, as further described hereinbelow.

Genomically multiplied plant seeds of the present invention can be generated using an improved method of colchicination, as described below.

Thus, according to an aspect of the invention, there is provided a method of generating a maize seed having a partially or fully multiplied genome, the method comprising contacting the maize seed with a G2/M cell cycle inhibitor under a transiently applied magnetic field thereby generating the maize seed having a partially or fully multiplied genome.

Typically, the G2/M cycle inhibitor comprises a microtubule polymerization inhibitor.

Examples of microtubule cycle inhibitors include, but are not limited colchicine, colcemid, trifluralin, oryzalin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

The G2/M inhibitor is comprised in a treatment solution which may include additional active ingredients such as antioxidants, detergents and histones.

While treating the seeds with a treatment solution which comprises the G2/M cycle inhibitor, the plant is further subjected to a magnetic field of at least 700 gauss (e.g., 1350 Gauss) for about 2 hr. The seeds are placed in a magnetic field chamber such as that described in Example 1. After the indicated time, the seeds are removed from the magnetic field.

To improve permeability of the seeds to the treatment solution, the seeds are subjected to ultrasound treatment (e.g., 40 KHz for 10 to 20 min) prior to contacting with the G2/M cycle inhibitor.

Wet seeds may respond better to treatment and therefore seeds can be soaked in an aqueous solution (e.g., distilled water) at the initiation of treatment.

According to a specific embodiment, the entire treatment is performed in the dark and at room temperature (about 23-26° C.) or lower [e.g., for the ultrasound (US) stage].

Thus according to a specific embodiment, the seeds are soaked in water at room temperature and then subjected to US treatment in distilled water.

Once permeated, the seeds are placed in a receptacle containing the treatment solution and a magnetic field in turned on. Exemplary ranges of G2/M cycle inhibitor concentrations are provided in Table 1 below. The treatment solution may further comprise DMSO, detergents, antioxidants and histones at the concentrations listed below.

Once the seeds are removed from the magnetic field they are subject to a second round of treatment with the G2/M cycle inhibitor. Finally, the seeds are washed and seeded on appropriate growth beds. Optionally, the seedlings are grown in the presence of Acadain™ (Acadian AgriTech) and Giberllon (the latter is used when treated with vinblastine, as the G2/M cycle inhibitor).

It will be appreciated that the above method may be implemented on the whole plant or plant part such as described herein and not necessarily restricted to seeds.

Using the above teachings, the present inventors have established genomically multiplied maize plants.

Once established, the plants of the present invention can be propagated sexually or asexually such as by using tissue culturing techniques.

As used herein the phrase “tissue culture” refers to plant cells or plant parts from which maize can be generated, including plant protoplasts, plant cali, plant clumps, and plant cells that are intact in plants, or part of plants, such as seeds, leaves, stems, straw, pollens, roots, root tips, anthers, ovules, petals, flowers, embryos, fibers and bolls.

According to some embodiments of the present invention, the cultured cells exhibit genomic stability for at least 2, 3, 4, 5, 7, 9 or 10 passages in culture.

Techniques of generating plant tissue culture and regenerating plants from tissue culture are well known in the art. For example, such techniques are set forth by Vasil., 1984. Cell Culture and Somatic Cell Genetics of Plants, Vol I, II, III, Laboratory Procedures and Their Applications, Academic Press, New York; Green et al., 1987. Plant Tissue and Cell Culture, Academic Press, New York; Weissbach and Weissbach, 1989. Methods for Plant Molecular Biology, Academic Press; Gelvin et al., 1990, Plant Molecular Biology Manual, Kluwer Academic Publishers; Evans et al., 1983, Handbook of Plant Cell Culture, MacMillian Publishing Company, New York; and Klee et al., 1987. Ann. Rev. of Plant Phys. 38:467 486.

The tissue culture can be generated from cells or protoplasts of a tissue selected from the group consisting of seeds, leaves, stems, pollens, roots, root tips, anthers, ovules, petals, flowers and embryos.

It will be appreciated that the plants of the present invention can also be used in plant breeding along with other maize plants (i.e., self-breeding or cross breeding) in order to generate novel plants or plant lines which exhibit at least some of the characteristics of the maize plants of the present invention.

Plants resultant from crossing any of these with another plant can be utilized in pedigree breeding, transformation and/or backcrossing to generate additional cultivars which exhibit the characteristics of the genomically multiplied plants of the present invention and any other desired traits. Screening techniques employing molecular or biochemical procedures well known in the art can be used to ensure that the important commercial characteristics sought after are preserved in each breeding generation.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as: Gressel., 1985. Biotechnologically Conferring Herbicide Resistance in Crops: The Present Realities, In: Molecular Form and Function of the plant Genome, L van Vloten-Doting, (ed.), Plenum Press, New York; Huftner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; and Koncz, C., et al. 1986, Molecular and General Genetics.

Inbreeding can be done by using techniques well known in the art. Typically, the seeds are recovered and planted. The resulting plants are then evaluated for the trait or traits being sought and those showing the desired traits are again self-pollinated and the seeds are harvested and planted. This process is repeated for sufficient number of generations until inbred lines having the desired traits are being developed. Such inbred lines are used to produce hybrid tetraploid or triploid maize.

It will be appreciated that plants or hybrid plants of the present invention can be genetically modified such as in order to introduce traits of interest e.g. enhanced resistance to stress (e.g., biotic or abiotic).

Thus, the present invention provides novel genomically multiplied plants and cultivars, and seeds and tissue culture for generating same.

The plant of the present invention is capable of self-breeding or cross-breeding with a diploid or tetraploid maize, or maize of various ploidies (e.g., induced high-ploidy maize as described herein) or with other maize species.

Thus, the present invention further provides for a hybrid plant having as a parental ancestor the genomically multiplied plant as described herein. Examples of hybrid triploids are provided hereinabove and in the Examples section which follows.

According to a specific embodiment the invention provides for a hybrid plant having as a parental ancestor the polyploid maize of the invention.

The present invention further provides for a seed bag which comprises at least 10%, 20% 50% or 100% of the seeds of the plants or hybrid plants of the invention.

The present invention further provides for a planted field which comprises any of the plants or hybrid plants of the invention.

The present invention further provides for a sown field which comprises any of the seeds of the plants or hybrid plants of the invention.

The present invention further contemplates products and processed products of the plants of parts thereof of the present invention.

As used herein a “processed product” refers to a maize plant of the invention, or parts thereof, that have undergone a mechanical or chemical change.

Examples of processed products include but are not limited to food, feed, herbal supplements, beverages, chemicals, construction material, biodiesel and biofuel.

According to a specific embodiment the product comprises cells of the plant or components thereof such as DNA, which can be qualitatively assessed for multiplication.

Any of the following products or uses, which constitute a non-limiting list, are contemplated by the present teachings. Cornflower, starch, meal and products thereof (e.g., polenta), pop-corn, hominy, silage, alcoholic beverages. Animal feed, baking snack foods, non-alcoholic beverages, building materials, canners/packers, cereals, chemicals. Condiments, confectionary, fats and oils, formulated dairy products, fuel alcohol, household needs, ice creams, frozen desserts, jams, jellies preserves, meat products, paper and related products, syrups and sweeteners, textile (clothing, carpeting, bedding), tobacco, polymer composites (HarvestForm™), compostable plastics, plastic foam, antifreeze, pharmaceuticals, host system for recombinant expression (e.g., vaccines, enzymes, extracellular matrix proteins)

The present invention also contemplates methods of producing the processed product or product.

For example there is provided a method of producing maize meal, the method comprising:

(a) harvesting grains of the plant of the invention; and

(b) processing the grains to produce the maize meal.

Alternatively, there is provided a method of producing oil, the method comprising:

(a) harvesting grains of the plant of the invention; and

(b) extracting oil from the grains.

It is expected that during the life of a patent maturing from this application many relevant products will be developed and the scope of the term processed product is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Polyploid Maize

Experimental Procedures

All stages were performed in the dark.

Seeds were soaked in a vessel full of water at about 25° C. for about 2 hr.

The seeds were transferred into a clean net bag and put into a distilled water-filled ultrasonic bath at about 23 about 26° C. Sonication was applied (about 40 KHz) for about 10 to about 20 minutes. Temperature was kept below 26° C. The seeds bag was placed in a vessel containing the treatment solution (described below) at about 25° C. The vessel was placed within the magnetic field chamber (described below) and incubated for about 2 hr. Seeds were removed from the bag and placed on top of a paper towel bed on a plastic tray. A second layer of paper towel soaked with treatment solution was used as a cover. The seeds were incubated for about 24-about 48 hr at about 25° C. and kept wet for the whole incubation period. The seeds were collected into a clean vessel and washed with water (pH=7). A seedling tray of soil supplemented with 25 ppm of 20:20:20 Micro Elements Fertilizer was prepared. Treated seeds were seeded to the tray and moved to nursery using a day temperature range of about 20-about 25° C., night range of about 10-about 17° C. and minimal moisture of about 40%.

When using Vinblastine, 0.5-1.5% GIBERLLON were applied immediately after seeding. The seeds were treated with ACADIAN™ twice a week for the following 3 weeks.

### Treatment Solution:

### DMSO 0.5%

TritonX100 5 drops/L
microtubule polymerization inhibitor

### Antioxidant

Histones 50-100 ug/ml
pH=6
Prepare in softened, nitrogen free water
*Use immediately.

TABLE 1

| | Concentration | |
|---|---|---|
| Microtubule polymerization inhibitor | | |
| Vinblastine sulphate | 0.05-0.2% | |
| Colchicine | 0.1-0.5 | mg/ml |
| Nocodazole | 0.1-0.9% | |
| Oryzalin | 0.002-0.005% | |
| Trifluralin | 0.002-0.005% | |
| Antioxidants | | |
| Cyanidin 3-O-b-glucopyranoside | 25-100 | ug/ml |
| Baicalein | 10⁻⁶⁻10⁻⁴ | M |
| Quercetin | 10⁻⁶⁻10⁻⁴ | M |
| Trolox | 5-10 | mM |

Magnetic Field Details:

The magnetic field chamber consisted of two magnet boards located 11 cm from each other. The magnetic field formed by the two magnets is a coil-shaped magnetic field with a minimal strength of 1350 gauss in its central axis. The seeds were placed in a net bag within a stainless steel bath filled with treatment solution (as described above), and the bath was inserted into the magnetic chamber.

### Example 2

### Triploid Hybrid Maize

Tetraploid males were generated according to the method of Example 1. The tetraploid males were used for triploid hybrid production. A diploid plant was used as the female parent in the crossings. The specifics of the crossings are described in Tables 2, 4 and 5 below.

Specifically, Table 2 relates to triploid plants having the N2-2011 as the female parent plant and Z1(12)-2011 or Z1(13)-2011 as the tetraploid male parent.

TABLE 2

| | Ploidy | Thousand seed | Male parent | Ploidy |
|---|---|---|---|---|
| codes | level | weight (gr) | code | level |
| Female parent code: | 2N | 168 | | |
| N2-2011 | | | | |
| HF1 code: EXPM 100 | 2N | 200.6 | Z1-2011 | 2N |
| HF1 code: EXPM 104 | 3N | 305 | Z1(13)-2011 | 4N |
| HF1 code: EXPM 110 | 3N | 343.65 | Z1(12)-2011 | 4N |

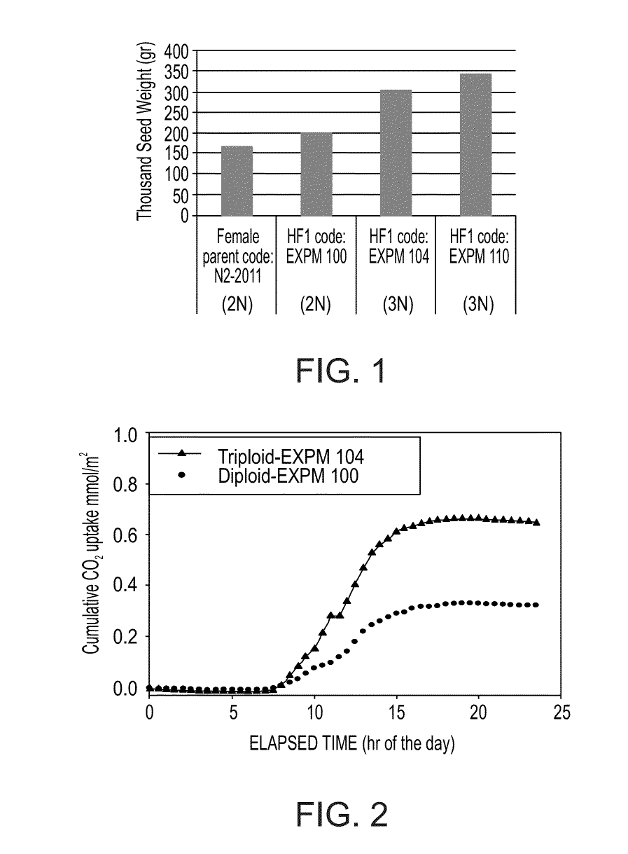

Grain weight and photosynthesis efficiency are illustrated in FIGS. 1 and 2 (the EXPM 100 EXPM 104 are demonstrated in FIG. 2).

Table 3 below summarizes the carbon dioxide uptake and dry matter production for the triploid hybrid versus the diploid hybrid having the same female parent.

TABLE 3

| | CO₂ uptake | Dry matter production |
|---|---|---|
| | (μmol/(m² day)) | (gr/(m2 day)) |
| Triploid- EXPM 104 | 0.65 | 19.7 |
| Diploid- EXPM 100 | 0.32 | 9.7 |

Table 4 below relates to triploid plants having the N2-2011 as the female parent plant and Z5(6)-2011 or Z5(8)-2011 as the tetraploid male parent.

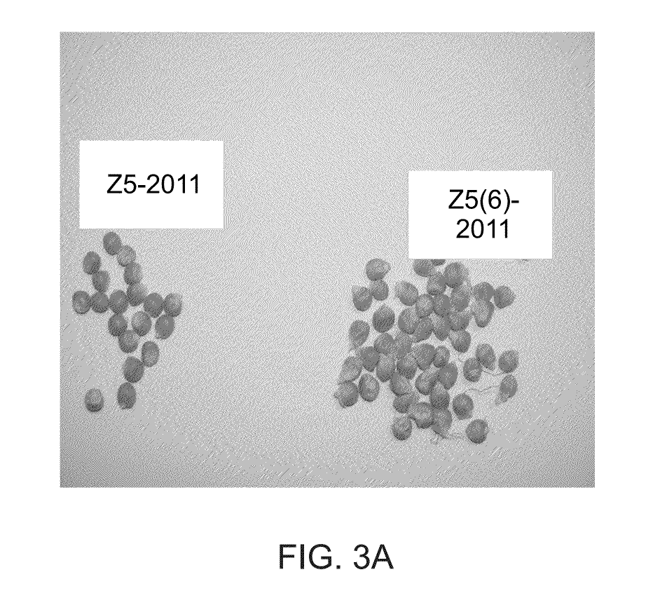

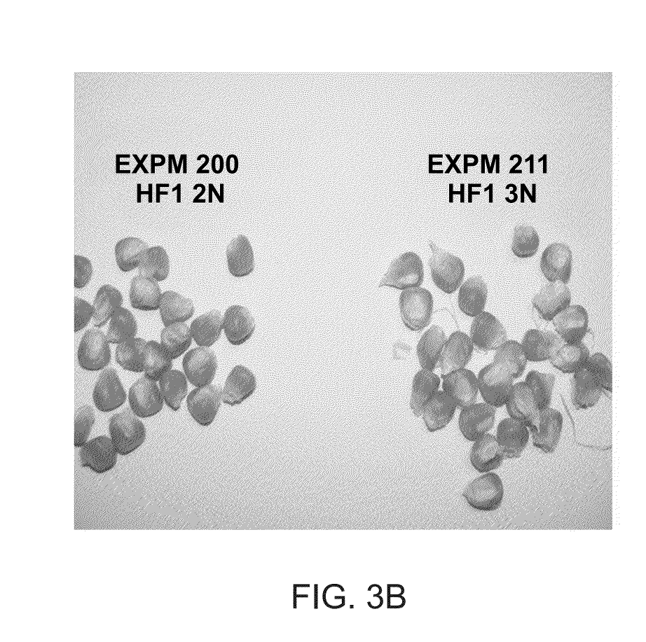

Table 4 and FIG. 3A-B provide structural features of hybrid triploid seeds versus those of the female diploid parent and male tetraploid parent.

TABLE 4

| | Hybrid seeds characteristics | | | Male Seeds characteristics | | | |
|---|---|---|---|---|---|---|---|
| | Ploidy | Thousand seed | | Male parent | Ploidy | Thousand seed | |
| Codes | Level | weight (gr) | Description | code | Level | weight (gr) | Description |
| Female parent | 2N | 168 | Small, | | | | |
| code: N2-2011 | | | sharpened, | | | | |
| | | | bright | | | | |
| | | | yellow | | | | |
| HF1 code: | 2N | 293.61 | Medium, | Z5- | 2N | 245.54 | Small, |
| EXPM 200 | | | Rounded, | 2011 | | | Rounded, |
| | | | Orange | | | | Bright orange |
| HF1 code: | 3N | 323.08 | Big and | Z5(6)- | 4N | 252.3 | Big, Rounded, |
| EXPM 208 | | | rounded, a | 2011 | | | orange with |
| | | | bit | | | | pigmentation |
| | | | grooved | | | | stains |
| HF1 code: | 3N | 329.29 | Very Big, | Z5(8)- | 4N | 262.55 | Big Rounded, |
| EXPM 211 | | | Orange, a | 2011 | | | dark orange |
| | | | bit | | | | with strong |
| | | | sharpened | | | | pigmentation |
| | | | | | | | stains |

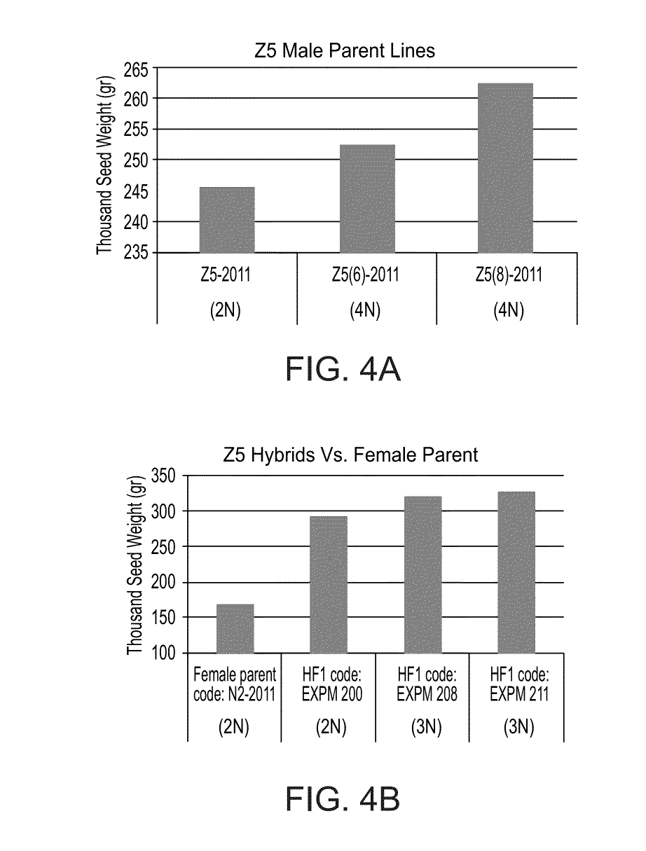

FIGS. 4A-B are graphs showing the relative weight of grains of diploid and tetraploid parents versus that of the triploid hybrid. Heterosis is displayed.

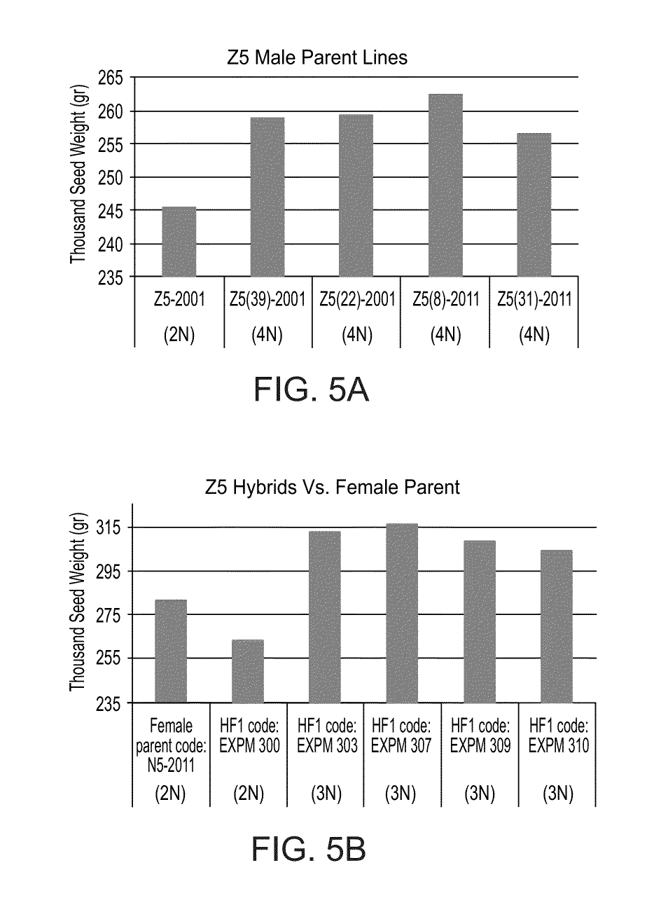

Table 5 below relates to triploid plants having the N5-2011 as the female parent plant and Z5(39)-2011, Z5(22)-2011, Z5(8)-2011 or Z5(31)-2011, as the tetraploid male parent. Table 5 further provides grain characteristics for the tetraploid male parents and triploid hybrids. FIGS. 5A-B show the relative weight (“thousand seed weight”) of the hybrid seeds.

TABLE 5

| Hybrid seeds characteristics | | | | Male Seeds characteristics | | | |
|---|---|---|---|---|---|---|---|
| | Ploidy | Thousand seed | | Male parent | Ploidy | Thousand seed | |
| | Level | weight (gr) | Description | code | Level | weight (gr) | Description |
| Female parent | 2N | 281.7 | Big, | | | | |
| code: N5-2011 | | | Sharpened, | | | | |
| | | | Bright | | | | |
| | | | Yellow | | | | |
| HF1 code: | 2N | 263.19 | Medium, | Z5- | 2N | 245.54 | Small, |
| EXPM 300 | | | sharpened, | 2011 | | | Rounded, |
| | | | yellow | | | | Bright |
| | | | | | | | orange |
| HF1 code: | 3N | 313.19 | Bigger, | Z5(39)- | 4N | 258.9 | Medium, |
| EXPM 303 | | | rounded, | 2011 | | | rounded |
| | | | darker. | | | | bright |
| | | | | | | | orange |
| HF1 code: | 3N | 316.67 | Medium, | Z5(22)- | 4N | 259.23 | Medium, |
| EXPM 307 | | | rounded, | 2011 | | | rounded |
| | | | Orange. | | | | bright |
| | | | Different. | | | | orange with |
| | | | | | | | pigmentation |
| | | | | | | | stains |
| HF1 code: | 3N | 309.31 | Medium, | Z5(8)- | 4N | 262.55 | Medium, |
| EXPM 309 | | | sharpened, | 2011 | | | very strong |
| | | | orange | | | | pigmentation |
| | | | | | | | stains |
| HF1 code: | 3N | 304.31 | Medium, | Z5(31)- | 4N | 256.65 | Medium |
| EXPM 310 | | | sharpened, | 2011 | | | rounded, |
| | | | orange | | | | orange |

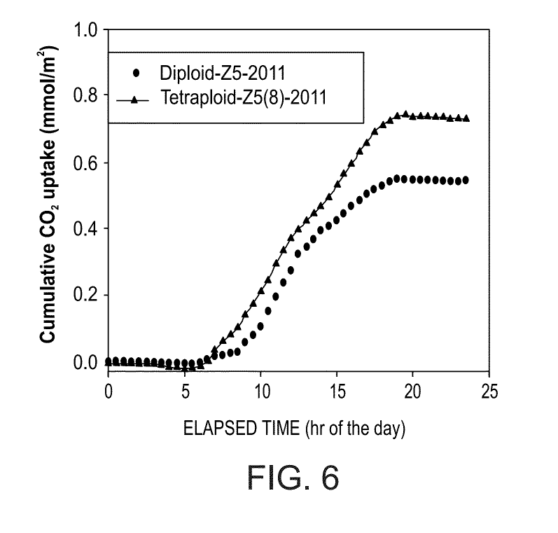

FIG. 6 shows the photosynthetic efficiency of the diploid male parent (Diploid-Z5-2011) and the teraploid plant (Z5(8)-2011).

Table 6 describes the total CO₂ uptake and dry matter production of the tetraploid Z5(8)-2011 (generated as described in Example 1) in comparison to the diploid isogenic plant.

TABLE 6

| | CO₂ uptake | Dry matter production |
|---|---|---|
| | (μmol/(m² day)) | (gr/(m2 day)) |
| Diploid- Z5-2011 | 0.54 | 16.2 |
| Tetraploid-Z5(8)-2011 | 0.73 | 21.8 |

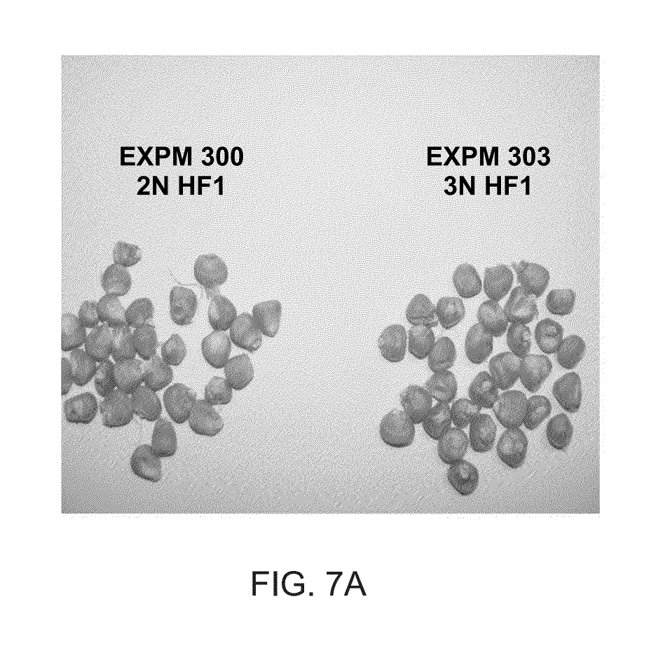

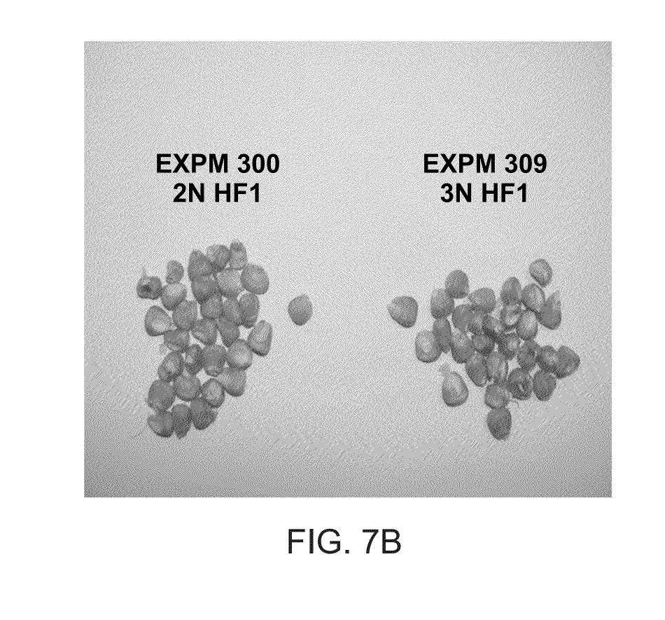

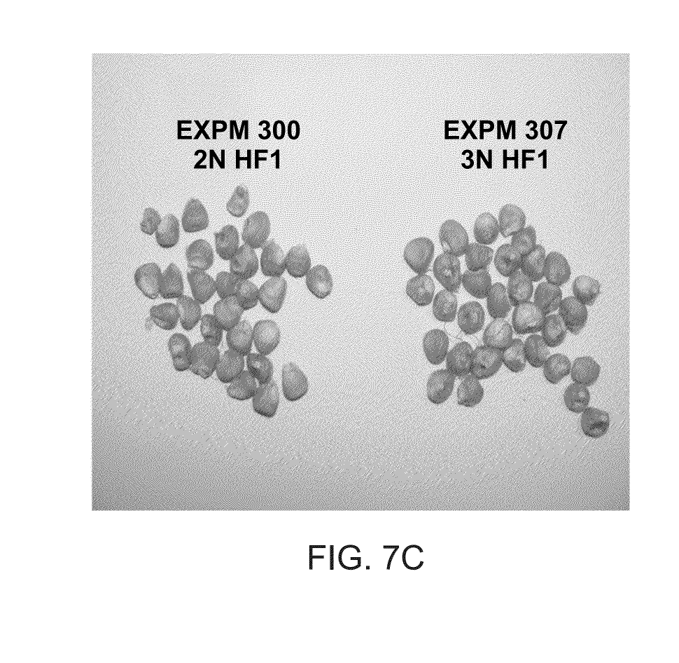

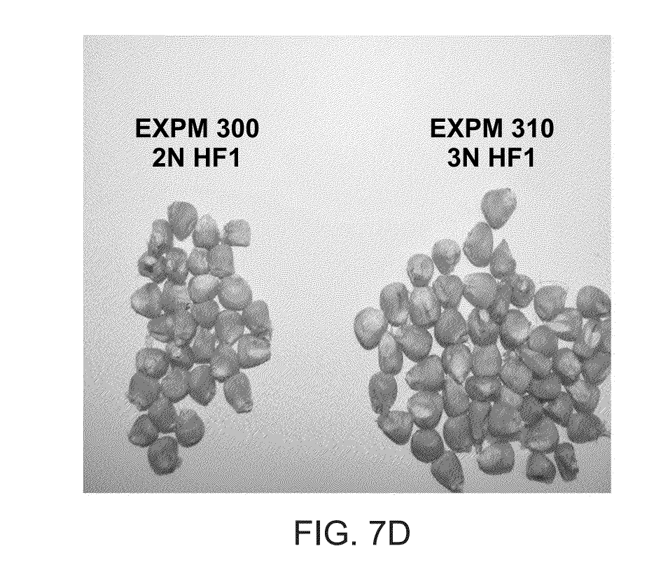

FIGS. 7A-B show the grains of a hybrid diploid versus a hybrid triploid maize, as described in details in Table 5 above.

The ploidy of male lines as evidenced by FACS is provided in Table 7 below.

TABLE 7

| Male line code | FACS Results | Ploidy Level |
|---|---|---|
| Z1-2011 | 300 | 2N |
| Z1(13)-2011 | 600 | 4N |
| Z5-2011 | 300 | 2N |
| Z5(6)-2011 | 600 | 4N |
| Z5(8)-2011 | 640 | 4N |
| Z5(31)-2011 | 640 | 4N |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-2. (canceled)

3. A maize plant having a partially or fully multiplied genome and characterized by a seed weight at least 10% higher than that of a diploid maize (Zea mays L. ssp) plant isogenic thereto, when being of the same developmental stage and when grown under the same conditions.

4. A maize plant having a partially or fully multiplied genome and characterized by a total dry weight least 30% higher than that of a diploid maize (Zea mays L. ssp) plant isogenic thereto when being of the same developmental stage and when grown under the same conditions.

5. A hybrid plant having as a parental ancestor the plant of claim 3.

6. A planted field comprising the plant of claim 3.

7. A sown filed comprising seeds of the plant of claim 3.

8. The plant of claim 4 having a seed weight at least 10% higher than that of said diploid maize (Zea mays L. ssp) plant isogenic thereto when being of the same developmental stage and when grown under the same conditions.

9. The plant of claim 3 having a total dry weight at least 30% higher than that of said diploid maize (Zea mays L. ssp) plant isogenic thereto when being of the same developmental stage and when grown under the same conditions.

10. The plant of claim 3, exhibiting higher CO₂ uptake than that of said diploid maize (Zea mays L. ssp) plant isogenic thereto when being of the same developmental stage and when grown under the same conditions.

11. The plant of claim 3, being at least as fertile as said diploid maize (Zea mays L. ssp) plant isogenic thereto when being of the same developmental stage and when grown under the same conditions.

12. The plant of claim 11, being of a first, second or third generation.

13. The plant of claim 3, being non-transgenic.

14. The plant of claim 11, wherein said fertility is determined by at least one of:
number of seeds per plant;
seed set assay;
gamete fertility assay; and
acetocarmine pollen staining.

15. The plant of claim 3, being a triploid.

16. The plant of claim 3, being a tetraploid.

17. The plant of claim 3 capable of cross-breeding with a diploid or tetraploid maize.

18. The hybrid of claim 5 being an inbred.

19. A plant part of the plant of claim 3.

20. The plant part of claim 19, being a seed or stover.

21. A processed product of the plant of claim 3.

22-23. (canceled)

24. A meal produced from the plant of claim 3.

25. A method of producing oil, the method comprising:
(a) harvesting grains of the plant of claim 3; and
(b) extracting oil from said grains.

26. The method of claim 25 further comprising processing said oil into biodiesel.

27. An isolated regenerable cell of the plant of claim 3.

28. The cell of claim 27, exhibiting genomic stability for at least 5 passages in culture.

29. The cell of claim 27 being from a meristem, a pollen, a leaf, a root, a root tip, an anther, a pistil, a flower, a straw, a seed or a stem.

30. A tissue culture comprising the regenerable cells of claim 27.

31. A method of producing maize seeds, comprising self-breeding or cross-breeding the plant of claim 3.

32. A method of developing a hybrid plant using plant breeding techniques, the method comprising using the plant of claim 3 as a source of breeding material for self-breeding and/or cross-breeding.

33. A method of producing maize meal, the method comprising:
(a) harvesting grains of the plant of claim 3; and
(b) processing said grains to produce the maize meal.

34. A method of generating a maize seed having a partially or fully multiplied genome, the method comprising contacting the maize seed with a G2/M cell cycle inhibitor under a transiently applied magnetic field thereby generating the maize seed having a partially or fully multiplied genome.

35. The method of claim 34, wherein said G2/M cell cycle inhibitor comprises a microtubule polymerization inhibitor.

36. The method of claim 35, wherein said microtubule polymerization inhibitor is selected from the group consisting of colchicine, nocodazole, oryzaline, trifluraline and vinblastine sulphate.

37. The method of claim 34 further comprises sonicating said seed prior to contacting.

38-39. (canceled)

40. A maize plant generated according to the method of claim 34.

41. The plant of claim 40, being of a first, second or third generation.

42. A maize plant having a partially or fully multiplied genome and characterized by grain yield per plant higher by 0.1-5, 0.3-5, 0.4-2.5, 1-5, 2-3 or 2-2.5 fold than that of a diploid maize (Zea mays L. ssp) plant isogenic thereto when being of the same developmental stage and when grown under the same conditions.

43. The plant of claim 42, being a tetraploid.

44. The plant of claim 42, being a triploid.

45. The plant of claim 42, characterized by at least 90% fertile pollen that are stained by acetocarmine; and alternatively or additionally at least 90% of seeds that germinate on sucrose.

46. A hybrid plant having as a parental ancestor the plant of claim 42.

47. The hybrid of claim 46, being an inbred.

48. The hybrid of claim 47, capable of crossing with a diploid maize plant to generate a fertile triploid maize plant.

49. The plant of claim 42, being of a first, second or third generation.

50. A maize plant having a partially or fully multiplied genome characterized by being at least as fertile as a diploid maize (Zea mays L. ssp) plant isogenic thereto when being of the same developmental stage and when grown under the same conditions.

51. The plant of claim 50, being a tetraploid.

52. The plant of claim 50, being a triploid.

53. The plant of claim 50, characterized by at least 90% fertile pollen that are stained by acetocarmine; and alternatively or additionally at least 90% of seeds that germinate on sucrose.

54. A hybrid plant having as a parental ancestor the plant of claim 50.

55. The hybrid of claim 54, being an inbred.

56. The hybrid of claim 55, capable of crossing with a diploid maize plant to generate a fertile triploid maize plant.

57. The plant of claim 50, being of a first, second or third generation.

58. A planted field comprising the plant of claim 42.

59. A sown filed comprising seeds of the plant of claim 42.

60. A planted field comprising the plant of claim 50.

61. A sown filed comprising seeds of the plant of claim 50.

62. A planted field comprising the plant of claim 4.

63. A sown filed comprising seeds of the plant of claim 4.

64. A meal produced from the plant of claim 50.

65. A method of producing oil, the method comprising:
(a) harvesting grains of the plant of claim 50; and
(b) extracting oil from said grains.

66. The method of claim 65 further comprising processing said oil into biodiesel.

67. An isolated regenerable cell of the plant of claim 50.

68. The cell of claim 67, exhibiting genomic stability for at least 5 passages in culture.

69. The cell of claim 67 being from a meristem, a pollen, a leaf, a root, a root tip, an anther, a pistil, a flower, a straw, a seed or a stem.

70. A tissue culture comprising the regenerable cells of claim 67.

71. A method of producing maize seeds, comprising self-breeding or cross-breeding the plant of claim 50.

72. A method of developing a hybrid plant using plant breeding techniques, the method comprising using the plant of claim 50 as a source of breeding material for self-breeding and/or cross-breeding.

73. A method of producing maize meal, the method comprising:
(a) harvesting grains of the plant of claim 50; and
(b) processing said grains to produce the maize meal.

74. A meal produced from the plant of claim 42.

75. A method of producing oil, the method comprising:
(a) harvesting grains of the plant of claim 42; and
(b) extracting oil from said grains.

76. The method of claim 75 further comprising processing said oil into biodiesel.

77. An isolated regenerable cell of the plant of claim 42.

78. The cell of claim 77, exhibiting genomic stability for at least 5 passages in culture.

79. The cell of claim 77 being from a meristem, a pollen, a leaf, a root, a root tip, an anther, a pistil, a flower, a straw, a seed or a stem.

80. A tissue culture comprising the regenerable cells of claim 77.

81. A method of producing maize seeds, comprising self-breeding or cross-breeding the plant of claim 42.

82. A method of developing a hybrid plant using plant breeding techniques, the method comprising using the plant of claim 42 as a source of breeding material for self-breeding and/or cross-breeding.

83. A method of producing maize meal, the method comprising:
(a) harvesting grains of the plant of claim 42; and
(b) processing said grains to produce the maize meal.

84. A maize plant having a partially or fully multiplied genome characterized by a grain yield per growth area at least as similar to that of a diploid maize (Zea mays L. ssp) plant isogenic thereto when being of the same developmental stage and when grown under the same conditions.

85. The plant of claim 84, being a tetraploid.

86. The plant of claim 84, being a triploid.

87. The plant of claim 84, characterized by at least 90% fertile pollen that are stained by acetocarmine; and alternatively or additionally at least 90% of seeds that germinate on sucrose.

88. A hybrid plant having as a parental ancestor the plant of claim 84.

89. The hybrid of claim 88, being an inbred.

90. The hybrid of claim 89, capable of crossing with a diploid maize plant to generate a fertile triploid maize plant.

91. The plant of claim 84, being of a first, second or third generation.

92. A planted field comprising the plant of claim 84.

93. A sown filed comprising seeds of the plant of claim 84.

94. A meal produced from the plant of claim 84.

95. A method of producing oil, the method comprising:
(a) harvesting grains of the plant of claim 84; and
(b) extracting oil from said grains.

96. The method of claim 95 further comprising processing said oil into biodiesel.

97. An isolated regenerable cell of the plant of claim 84.

98. The cell of claim 97, exhibiting genomic stability for at least 5 passages in culture.

99. The cell of claim 97 being from a meristem, a pollen, a leaf, a root, a root tip, an anther, a pistil, a flower, a straw, a seed or a stem.

100. A tissue culture comprising the regenerable cells of claim 97.

101. A method of producing maize seeds, comprising self-breeding or cross-breeding the plant of claim 84.

102. A method of developing a hybrid plant using plant breeding techniques, the method comprising using the plant of claim 84 as a source of breeding material for self-breeding and/or cross-breeding.

103. A method of producing maize meal, the method comprising:
(a) harvesting grains of the plant of claim 84; and
(b) processing said grains to produce the maize meal.

104. The plant of claim 3, characterized by an increased abiotic stress tolerance as compared to said diploid plant isogenic thereto.

105. The plant of claim 4, characterized by an increased abiotic stress tolerance as compared to said diploid plant isogenic thereto.

106. The plant of claim 42, characterized by an increased abiotic stress tolerance as compared to said diploid plant isogenic thereto.

107. The plant of claim 50, characterized by an increased abiotic stress tolerance as compared to said diploid plant isogenic thereto.

108. The plant of claim 84, characterized by an increased abiotic stress tolerance as compared to said diploid plant isogenic thereto.
